(19) United States
(12) Patent Application Publication
(10) Pub. No.: US 2004/0223934 A1
(43) Pub. Date: Nov. 11, 2004

(54) Soluble skin layer, scalp with hair and model head made thereof

(76) Inventors: Chengkuo Chao, Changqing Borough Jinan City (CN); Shuping Qiao, Changqing Borough Jinan City (CN); Bingkun Ou, Changqing Borough Jinan City (CN)

Correspondence Address: SUMMA & ALLAN, P.A., 11610 NORTH COMMUNITY HOUSE ROAD, SUITE 200, CHARLOTTE, NC, 28277, US

(21) Appl. No.: 10/872,247

(22) Filed: Jun. 18, 2004

Related U.S. Application Data
(63) Continuation-in-part of application No. PCT/CN03/00313, filed on Apr. 28, 2003.

(30) Foreign Application Priority Data
May 23, 2002 (CN) 02110446.8

(51) Int. Cl. A61K 7/06; A61K 7/11

(52) U.S. Cl. 424/70.11

(57) ABSTRACT

This invention relates to a soluble skin layer, a scalp with hair made of the skin layer, and a model head with changeable skin, which is made of the skin layer or the scalp with hair. The soluble skin layer is formed by a water-soluble adhesive which is made of film forming material, plasticizer, defoaming agent, and cosolvent. The soluble skin layer can be used to make a scalp with hair having a soluble skin layer by implanting hair on the surface thereof. A model head which can be used for hairdressing training can be made by combining the scalp with hair with a support unit. The skin layer can be dissolved in water after use and recycled, which can provide environment protection and lower the cost of a model head.

### CROSS-REFERENCE TO RELATED APPLICATION(S)

This application is a continuation of PCT Application No. PCT/CN03/00313, filed Apr. 28, 2003, which designates the U.S., which is hereby incorporated herein by reference in its entirety, and which claims priority from Chinese Application No. 02110446.8, filed May 23, 2002.

### FIELD OF THE INVENTION

The present invention relates to in stage props or as a teaching tool of hair. Specifically, it relates to a soluble skin layer, a scalp with hair made of the skin layer, and a model head with replaceable scalps made of the skin layer or scalp with hair.

### BACKGROUND OF THE INVENTION

Model heads with hair are generally required in large numbers for cosmetology training for students studying hairdressing. Generally, hair on a model head is planted directly in a PVC material forming a skin layer, and polyurethane foam material is poured into the skin layer to form the whole model head. Cosmetology training typically includes styling and trimming hair, so when the model's “scalp” is cut short or shaved clean, the model can no longer be used. At present, conventional practice is to throw out used model heads.

The one-time use of model heads is a waste of resources, and the process for producing whole model heads is expensive. The one-time use of model heads results in large size waste, while the disposal of large amounts of high molecular materials can be undesirable from an environmental pollution. The investment costs for the treatment and reuse of these materials is high as well.

### SUMMARY OF THE INVENTION

The inventors have developed a feasible method for lowering costs and increasing usage efficiency for model heads. This in turn can improve the economies of using such products as well as minimize or eliminate adverse environmental affects resulting from the disposal of such products.

The present invention provides a soluble skin layer made from nontoxic water-soluble components. The soluble skin layer can be destroyed after use by directly dissolving it in water, thus avoiding the production of environmental pollution. The water solution can be recycled as well.

The water-soluble skin layer is formed from a water-soluble adhesive containing the following components: a film forming material, plasticizer, defoaming agent and cosolvent.

The soluble skin layer can be used on its own. For example, as a stage prop for applying cosmetics, among other uses, and can be dissolved directly in water afterward. With hair planted on its surface it works as a soluble scalp with hair. Thus, the present invention also provides a type of scalp with hair, wherein hair is planted on the surface of the soluble skin layer. The scalp can also be dissolved in water in the same way, or reused.

The present invention also provides a type of model head with a replaceable scalp, including a scalp with hair and support components, for use in cosmetology training. The scalp with hair can be attached to an outer part of a support, for example a support in the shape of the head of a human, to make a complete model head. After use the scalp can be removed and disposed of, and a new scalp can be placed onto the support in its place, thus reducing training costs. The invention can also provide benefits regarding environmental protection as well.

### BRIEF DESCRIPTION OF THE DRAWINGS

Having thus described the invention in general terms, reference will now be made to the accompanying drawings, which are not necessarily drawn to scale, and wherein:

FIG. 1 is a schematic illustration of a scalp with hair in accordance with the present invention; and

FIG. 2 is a schematic illustration of a model head in accordance with the present invention.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The present invention provides a soluble skin layer using a water soluble adhesive formed by the following components: a film forming material at approximately 6-10 parts by weight; a plasticizer at approximately 0.6-5.8 parts by weight; a defoaming agent at approximately 0.2-0.8 parts by weight; and a cosolvent at approximately 0.2-1.2 parts by weight.

Based upon the components given above, the present invention provides a composition with good film forming properties and which is water-soluble. When placed in 30 to 80 parts water, a water-soluble adhesive is produced with good film forming properties.

With regard to the raw materials given above, the film forming material can be chosen from one or more of polyvinyl alcohol, carboxyl-methyl cellulose and oxidized starch or a mixture thereof. The plasticizer can be chosen from glycerine and polyethylene glycol (preferably with an average molecular weight at a low of between 200 and 500). The defoaming agent can be chosen from tributyl phosphate or polyethylene glycol. The cosolvent can be a vinyl silane coupling agent, such as vinyltris (β-methoxy-ethoxy) silane, which is used to improve the water solubility of the primary raw materials. The raw materials used in the present invention are advantageously chemical substances which are not harmful to humans and that come from basic chemical materials. Appropriate amounts of essence and dye may be optionally added where required.

In advantageous aspects of the present invention, the composition and proportions by weight of the water soluble adhesive may be as follows: polyvinyl alcohol 6.0-8.0, water 40-50, glycerine 3.5-5.0, tributyl phosphate 0.4-0.6 or polyethylene glycol 0.2-0.8, and vinyl silane 0.6-0.7.

Based upon the composition given above, the alcoholysis rate of the polyvinyl alcohol is preferably between 87 and 89%. The average molecular weight of the polyethylene glycol is preferably in the range of 200-500. The vinyl silane can be vinyltris (β-methoxy-ethoxy) silane.

In the composition given above, polyethylene glycol can function both as a plasticizer and as a defoaming agent. When choosing such substances, the amount used can be modified within the parameters indicated above depending upon various factors, including the particular requirements for a given application.

The method for preparing the soluble skin layer is as follows:

The raw materials are heated in their prescribed proportions to their dissolution temperature to produce a consistent water-soluble adhesive;

The resultant water-soluble adhesive is dried after being formed on the surface of a mold.

During the preparation of the water soluble adhesive appropriate heating temperatures should be used based upon the special properties of the raw materials, i.e. the temperature should be at the point where the material dissolves so that it attains a consistent state. For example, the compositions noted above can be heated to about between 50 and 85° C.

Depending upon different requirements, the adhesive can be made to form on the surface of the mold. Due to its film forming action, the adhesive is able to form into a film very quickly and after drying and removal becomes a soluble skin layer. The drying process can optionally be repeated multiple times to satisfy requirements for thickness and strength. The forming process can be accomplished using any feasible operation, such as a brush application or pouring. After the adhesive film has dried on the surface of the mold, it is removed and can be placed in an oven to be dried.

The method given above conveniently provides the production of a skin layer which can be dissolved in water. By planting hair on the surface of the skin layer a scalp with hair can be made, which also can be dissolved in water for disposal after use.

According to an another aspect of the present invention, it provides a scalp with hair and a model head with hair on which scalps can be interchanged.

Based upon practical use requirements, the thickness of the skin layer on the scalp with hair can be about between 0.7 and 0.8 mm. A suitable waterproof membrane can also be optionally applied to the surface of the skin layer.

A method for preparing the product can include the following steps:

The raw materials are heated together in their prescribed proportions to their dissolution temperature to form a consistent water soluble adhesive;

The water-soluble adhesive is dried after being formed on the surface of a mold, and the process can optionally be repeated as necessary to obtain the required thickness for the skin layer;

After hair is planted on the surface of the skin layer, the roots of the hair on an inner side of the layer are trimmed.

With regard to the method given above, the temperature for heating the water soluble adhesive during preparation can be approximately 50-85° C. Because hair is planted on the surface of the skin layer, after planting, the roots on the inner side of the layer need to be trimmed. This process includes the following steps: the root part of the hair is shaved off; hot-melt latex is applied with a brush; and then heat pressure is applied to flatten the roots. Before planting the hair a waterproof membrane layer can be applied to the surface of the skin layer. There are no special requirements for the components of the membrane. For example, a polyurethane waterproof membrane can be applied with a brush.

After it is prepared, the scalp with hair is placed around the outside of a suitable support component to become a model head, allowing students to practice hairstyling. The support can be designed in the form of a human head. After the scalp with hair being assembled, a model head may be formed which is the same as those of prior art.

It can be seen from the description given above that the present invention provides a model head with a replaceable scalp. This can reduce both costs for model heads and stress upon the environment. The present invention thus can provide an economical and practical solution and advantages to the problems associated with conventional model heads.

The present invention provides generally the following benefits:

When using the model head with hair for training hairstylists, the replaceable scalp with hair on a model head can be fixed to model skulls used for this purpose or common model heads using connectors. Once use of the hair is completed, the scalp can be removed, and a new scalp with hair can be affixed to the skull or model head. Because the scalp is made from a soluble film forming material, used scalps can be soaked in water to dissolve the same. They are thus easily treated and can be recycled and reused. Thus, the present invention can reduce usage of skull materials on model heads, and thereby save resources, lower costs, and benefit the environment. In addition, the replaceable scalp of the invention can be easily shipped and stored, thus reducing transportation costs.

The following examples are given to illustrate the invention but should not be considered in limitation of the invention.

Embodiment 1

The soluble skin layer may be composed of the following raw materials in their respective parts by weight:

Polyvinyl alcohol (87-89% alcoholysis rate):7.5; water, 45; glycerine:4.8; tri-butyl phosphate:0.6; vinyltris (β-methoxy-ethoxy) silane:0.8.

Embodiment 2

The soluble skin layer may be composed of the following raw materials in their respective parts by weight:

Polyvinyl alcohol (87-89% alcoholysis rate) or carboxyl-methyl cellulose:6; water:35; glycerine or polyethylene glycol:3.0; polyethylene glycol:0.6; vinyltris (β-methoxy-ethoxy)silane:0.6.

Embodiment 3

The soluble skin layer may be composed of the following raw materials in their respective parts by weight:

Polyvinyl alcohol (87-89% alcoholysis rate): 10; water:60; glycerine:5.0; tri-butyl phosphate:0.8; vinyltris (β-methoxy-ethoxy) silane:1.1.

Embodiment 4

The soluble skin layer may be composed of the following raw materials in their respective parts by weight:

Polyvinyl alcohol (87-89% alcoholysis rate):8; water, 45; glycerine:1.0; tri-butyl phosphate:0.6; vinyltris (β-methoxy-ethoxy) silane:0.3.

Embodiment 5

The soluble skin layer may be composed of the following raw materials in their respective parts by weight:

Polyvinyl alcohol (87-89% alcoholysis rate):4; carboxyl-methyl cellulose:2; water, 35; polyethylene glycol:3.5; vinyltris(β-methoxy-ethoxy)silane:0.6.

Embodiment 6

The raw materials are mixed together in the proportions given in Embodiments 1 to 5. A small amount of dye and essence are added. The mixture is heated and dissolved at 50-80° C., and then stirred for between 2 and 2.5 hours to make the water-soluble adhesive.

The adhesive is then poured into a pour mold and dried in an oven. This process may be repeated multiple times until the thickness of the scalp reaches 0.7-0.8 mm. A polyurethane waterproof membrane is applied on the surface, and the product is then removed from the mold.

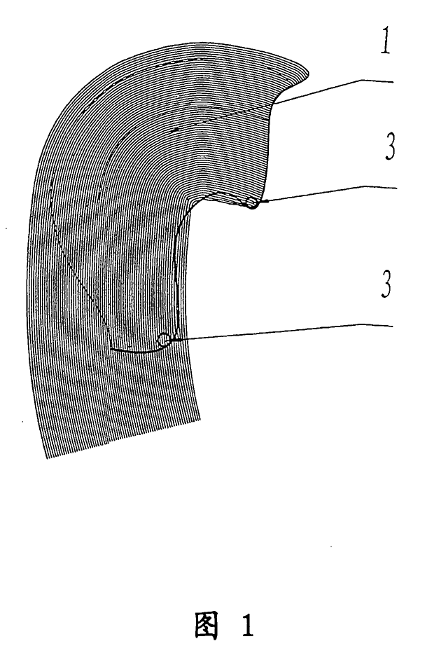

Hair is planted on the surface of the skin layer using a conventional method. The scalp is then turned over, and the roots of the hair are trimmed off with hair clippers. A layer of water-soluble hot melt polyvinyl acetate latex is brushed on the surface to adhere the hair. Heat pressure is then used to flatten the roots, and the result is the scalp with hair shown in FIG. 1.

In FIG. 1, the scalp with hair is designated as 1; and a connector is designated as 3.

Embodiment 7

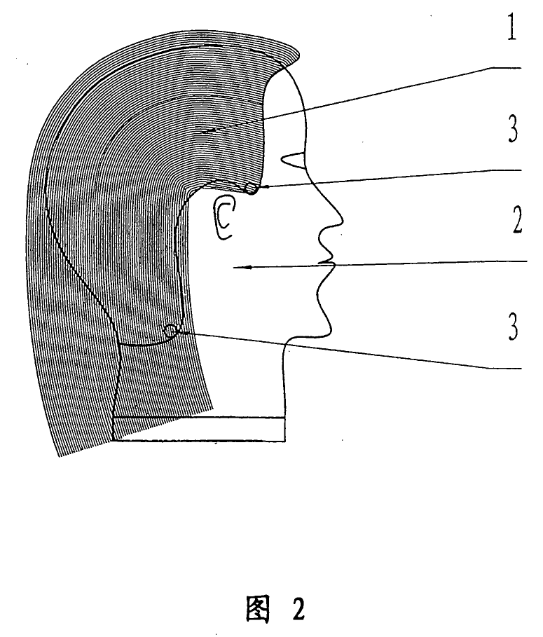

The scalp with hair of Embodiment 6 is placed over a skull to make the model head. As shown in FIG. 2, wherein the skull (support component) is designated as 2, connector 3, which fixes the scalp to skull 2, is attached along the edge of the scalp using any well-known method, such as non-drying adhesive, snap fasteners or connector pins.

The model head can be used in the same fashion as common products. After use the connector pins are unfastened and the scalp is removed and placed into water. The skin layer gradually dissolves, clearing up the polyurethane waterproof membrane and any fallen hair. The water solution can be recycled and reused. Skull 2 can be reused once a new scalp is adhered to it.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed is:

1. A soluble skin layer formed by a water-soluble adhesive composition comprising:
6 to 10 parts by weight of one or more film forming materials;
30 to 80 parts by weight water;
0.6 to 5.8 parts by weight of one or more plasticizers;
0.2 to 0.8 parts by weight of one or more defoaming agents; and
0.2 to 1.2 parts by weight of one or more cosolvents.

2. The soluble skin layer of claim 1, wherein:
one or more of said film forming materials comprise one or more of polyvinyl alcohol, carboxyl-methyl cellulose and oxidized starch;
one or more of said plasticizers comprise glycerine or polyethylene glycol or both;
one or more of said defoaming agents comprise tri-butyl phosphate or polyethylene glycol or both; and
one or more of said cosolvents comprises vinyl silane.

3. The soluble skin layer of claim 2, wherein the composition comprises:
6.0 to 8.0 parts by weight polyvinyl alcohol;
40 to 50 parts by weight water;
3.5 to 5.0 parts by weight glycerine;
0.4 to 0.6 parts by weight tri-butyl phosphate; and
0.6 to 0.7 parts by weight vinyl silane.

4. The soluble skin layer of claim 2, wherein the composition comprises:
6.0 to 8.0 parts by weight polyvinyl alcohol;
40 to 50 parts by weight water;
3.5 to 5.0 parts by weight glycerine;
0.2 to 0.8 parts by weight polyethylene glycol; and
0.6 to 0.7 parts by weight vinyl silane.

5. The soluble skin layer of claim 2, wherein the vinyl silane is vinyltris (β-methoxy-ethoxy) silane.

6. The soluble skin layer of claim 2, wherein the alcoholysis rate of the polyvinyl alcohol is between 87 and 89%.

7. A method for preparing a soluble skin layer, comprising the steps of:
mixing 6 to 10 parts by weight of one or more film forming materials; 30 to 80 parts by weight water; 0.6 to 5.8 parts by weight of one or more plasticizers; 0.2 to 0.8 parts by weight of one or more defoaming agents; and 0.2 to 1.2 parts by weight of one or more cosolvents to form a composition;
heating the composition under conditions sufficient to dissolve the components thereof and to form a consistent water soluble adhesive;
applying the water soluble adhesive to a surface of a mold; and
drying the adhesive to form a soluble skin layer.

8. The method of claim 7, wherein:
one or more of said film forming materials comprise one or more of polyvinyl alcohol, carboxyl-methyl cellulose and oxidized starch;
one or more of said plasticizers comprise glycerine or polyethylene glycol or both;
one or more of said defoaming agents comprise tri-butyl phosphate or polyethylene glycol or both; and
one or more of said cosolvents comprises vinyl silane.

9. The method of claim 7, wherein the composition comprises:
6.0 to 8.0 parts by weight polyvinyl alcohol;
40 to 50 parts by weight water;
3.5 to 5.0 parts by weight glycerine;
0.4 to 0.6 parts by weight tri-butyl phosphate; and
0.6 to 0.7 parts by weight vinyl silane.

10. The method of claim 7, wherein the composition comprises:
6.0 to 8.0 parts by weight polyvinyl alcohol;
40 to 50 parts by weight water;
3.5 to 5.0 parts by weight glycerine;
0.2 to 0.8 parts by weight polyethylene glycol; and
0.6 to 0.7 parts by weight vinyl silane.

11. The method of claim 7, wherein said heating step comprises heating the composition at a temperature of about 50-85° C.

12. The method of claim 7, further comprising repeating said applying and drying steps to form a skin layer having a required thickness.

13. A scalp with hair formed by planting hair in the surface of a soluble skin layer, wherein said soluble skin layer is made from a water-soluble adhesive composition comprising:
6 to 10 parts by weight of one or more film forming materials;
30 to 80 parts by weight water;
0.6 to 5.8 parts by weight of one or more plasticizers;
0.2 to 0.8 parts by weight of one or more defoaming agents; and
0.2 to 1.2 parts by weight of one or more cosolvents.

14. The scalp with hair of claim 13, wherein the thickness of said skin layer is between about 0.7 and 0.8 mm.

15. The scalp with hair of claim 13, further comprising a waterproof membrane applied to a surface of said skin layer.

16. A method for making a scalp with hair, comprising:
mixing 6 to 10 parts by weight of one or more film forming materials; 30 to 80 parts by weight water; 0.6 to 5.8 parts by weight of one or more plasticizers; 0.2 to 0.8 parts by weight of one or more defoaming agents; and 0.2 to 1.2 parts by weight of one or more cosolvents to form a composition;
heating the composition under conditions sufficient to dissolve the components thereof and to form a consistent water soluble adhesive;
applying the water soluble adhesive on a surface of a mold;
drying the adhesive to form a soluble skin layer;
optionally repeating the applying and heating steps to form a skin layer having a required thickness;
planting hair on a surface of the skin layer; and
optionally trimming the roots of the hair on the opposing surface of the skin layer.

17. The method of claim 16, further comprising applying a waterproof membrane layer to the surface of the completed skin layer.

18. The method of claim 16, wherein said heating step is conducted at a temperature of about 50 to 85° C. during mixing of the soluble adhesive.

19. The method of claim 16, wherein said trimming step comprises trimming the roots on the inside of the skin layer, applying hot melt latex with a brush, and applying heat pressure to flatten the roots.

20. A model head with interchangeable scalps, comprising a scalp with hair and one or more support components, wherein the scalp with hair is adhered to an outer portion of a support component, and wherein said scalp with hair is made by planting hair on a surface of a soluble skin layer made from a water soluble adhesive comprising 6 to 10 parts by weight of one or more film forming materials; 30 to 80 parts by weight water; 0.6 to 5.8 parts by weight of one or more plasticizers; 0.2 to 0.8 parts by weight of one or more defoaming agents; and 0.2 to 1.2 parts by weight of one or more cosolvents.

21. The model head of claim 20, wherein said support component is in the form of a human head.
